(19) United States
(12) Patent Application Publication
(10) Pub. No.: US 2025/0050082 A1
(43) Pub. Date: Feb. 13, 2025

(54) DEVICES, ASSEMBLIES, AND METHODS FOR DELIVERING A FLUID PREPARATION INTO A GASTROINTESTINAL TRACT

(71) Applicant: Rani Therapeutics, LLC, San Jose, CA (US)

(72) Inventors: Mir IMRAN, Los Altos Hills, CA (US); Arthur Hsu Chen CHANG, Irvine, CA (US); Varghese K. GEORGE, San Antonio, TX (US); Charles Gregory NELSON, New Braunsfels, TX (US); Paul SPEHR, San Antonio, TX (US)

(73) Assignee: Rani Therapeutics, LLC, San Jose, CA (US)

(21) Appl. No.: 18/810,252

(22) Filed: Aug. 20, 2024

Related U.S. Application Data
(63) Continuation of application No. PCT/US2023/013382, filed on Feb. 18, 2023.
(60) Provisional application No. 63/312,355, filed on Feb. 21, 2022.

(51) Int. Cl. A61M 31/00; A61M 39/22

(52) U.S. Cl. CPC A61M 31/002; A61M 39/221

(57) ABSTRACT

A delivery assembly for an ingestible device for delivering a fluid preparation into a GI lumen wall or surrounding tissue of a subject includes a housing, a piston-needle assembly, a membrane, and a valve member. The housing defines a chamber. The piston-needle assembly is movably disposed in the chamber and includes an inlet and a channel. The membrane is coupled to the housing such that the membrane and the housing cooperatively define a reservoir for containing the fluid preparation. The housing further defines an opening extending between the chamber and the reservoir. In response to sufficient movement of the piston-needle assembly relative to the housing, the valve member opens to allow the fluid preparation to flow from the reservoir through the opening into the chamber where the fluid preparation is directed to the inlet and the channel for delivery into the GI lumen wall or surrounding tissue thereof.

### CROSS-REFERENCE TO RELATED APPLICATIONS

This application claims the benefit of and priority to U.S. Provisional Application No. 63/312,355, filed on Feb. 21, 2022, the entire contents of which are hereby incorporated by reference herein.

### BACKGROUND

A therapeutic agent such as a drug may be administered to a subject by ingestion or through parenteral injection (e.g., subcutaneously, intramuscularly, or intravenously) to provide a desired therapeutic effect. However, these routes of administration have some disadvantages. For example, some therapeutic agents such as large (macro) molecules are not suitable for delivery by ingestion because of enzymatic breakdown of these molecules in the gastrointestinal (GI) tract of a subject. Other types of therapeutic agents may otherwise be poorly tolerated within the GI tract resulting in low systemic uptake. With parenteral injections, subjects may experience pain and inconvenience with administration which can significantly impact compliance and quality of life.

To address these disadvantages, ingestible devices have been proposed to orally deliver a solid form of a therapeutic agent into a lumen wall or surrounding tissue of the GI tract. However, solid dosage forms may be limited to relatively small dosages (e.g., a few milligrams). Thus, using solid dosage forms may be disadvantageous for therapy regimens that require higher dosages due to, for example, frequency of dosing, convenience, or cost.

Accordingly, there is a need for devices which can deliver a broad range of dosages of a therapeutic agent and can address one or more of the disadvantages associated with conventional oral and parenteral routes of administration.

### SUMMARY

Embodiments of the present disclosure relate generally to devices, assemblies, and methods for delivering a fluid preparation into the GI tract of a subject (e.g., into a GI lumen wall or surrounding tissue). The devices and assemblies are structured to deliver up to about 400 microliters (μl) or more of a fluid preparation, which may include a dosage of up to about 100 mg or more of at least one therapeutic agent. In this manner, the disclosed devices, assemblies, and methods can allow for efficient delivery of a broad range of dosages of therapeutic agents for most therapeutic regimens.

In one aspect, a delivery assembly for an ingestible device for delivering a fluid preparation into a GI lumen wall or surrounding tissue of a subject includes a housing, a piston-needle assembly, a membrane, and a valve member. The housing defines a chamber. The piston-needle assembly is movably disposed in the chamber and includes an inlet and a channel. The membrane is coupled to the housing such that the membrane and the housing cooperatively define a reservoir for containing the fluid preparation. The housing further defines an opening extending between the chamber and the reservoir. The valve member selectively controls a flow of the fluid preparation from the reservoir to the chamber through the opening. In response to sufficient movement of the piston-needle assembly relative to the housing, the valve member opens to allow the fluid preparation to flow from the reservoir through the opening into the chamber. The fluid preparation is directed from the chamber to the inlet and the channel for delivery into the GI lumen wall or surrounding tissue thereof.

In another aspect, an ingestible device for delivering a fluid preparation into a GI lumen wall or surrounding tissue of a subject includes an expandable member and a delivery assembly coupled to, and disposed in, the expandable member. The delivery assembly includes a housing, a piston-needle assembly, a membrane, a fluid preparation, and a valve member. The housing defines a chamber. The piston-needle assembly is movably disposed in the chamber and includes an inlet and a channel. The membrane is coupled to the housing such that the membrane and the housing cooperatively define a reservoir. The housing further defines an opening extending between the chamber and the reservoir. The fluid preparation is disposed in the reservoir and includes at least one therapeutic agent. The valve member selectively controls a flow of the fluid preparation from the reservoir to the chamber through the opening. In response to sufficient movement of the piston-needle assembly relative to the housing, the valve member opens to allow the fluid preparation to flow from the reservoir through the opening into the chamber. The fluid preparation is directed from the chamber to the inlet and the channel for delivery into the GI lumen wall or surrounding tissue thereof.

In another aspect, a method for delivering a fluid preparation into a GI lumen wall or surrounding tissue of a subject in need thereof includes ingesting, by the subject, an ingestible device as described herein. The ingestible device includes an expandable member and a delivery assembly coupled to, and disposed in, the expandable member. The delivery assembly includes a housing, a piston-needle assembly, a membrane, a fluid preparation, and a valve member. The housing defines a chamber. The piston-needle assembly is movably disposed in the chamber and includes an inlet and a channel. The membrane is coupled to the housing such that the membrane and the housing cooperatively define a reservoir. The housing further defines an opening extending between the chamber and the reservoir. The fluid preparation is disposed in the reservoir and includes at least one therapeutic agent. The valve member selectively controls a flow of the fluid preparation from the reservoir to the chamber through the opening. In response to sufficient movement of the piston-needle assembly relative to the housing, the valve member opens to allow the fluid preparation to flow from the reservoir through the opening into the chamber. The fluid preparation is directed from the chamber to the inlet and the channel for delivery into the GI lumen wall or surrounding tissue thereof.

In another aspect, use of an ingestible device for delivering a fluid preparation into a GI lumen wall or surrounding tissue of a subject in need thereof includes ingesting, by the subject, an ingestible device as described herein. The ingestible device includes an expandable member and a delivery assembly coupled to, and disposed in, the expandable member. The delivery assembly includes a housing, a piston-needle assembly, a membrane, a fluid preparation, and a valve member. The housing defines a chamber. The piston-needle assembly is movably disposed in the chamber and includes an inlet and a channel. The membrane is coupled to the housing such that the membrane and the housing cooperatively define a reservoir. The housing further defines an opening extending between the chamber and the reservoir. The fluid preparation is disposed in the reservoir and includes at least one therapeutic agent. The valve member selectively controls a flow of the fluid preparation from the reservoir to the chamber through the opening. In response to sufficient movement of the piston-needle assembly relative to the housing, the valve member opens to allow the fluid preparation to flow from the reservoir through the opening into the chamber. The fluid preparation is directed from the chamber to the inlet and the channel for delivery into the GI lumen wall or surrounding tissue thereof.

In another aspect, there are provided methods of preparing an ingestible device for delivering a therapeutic agent into a GI lumen wall or surrounding tissue of a subject, comprising filling a fluid preparation comprising a therapeutic agent into a delivery assembly as described herein.

In one or more embodiments of any of the foregoing aspects, the piston-needle assembly and the housing cooperatively define a fluid channel within the chamber for directing the fluid preparation from the opening to the inlet.

In one or more embodiments of any of the foregoing aspects, the valve member includes a plug detachably coupled to the housing at the opening. In one or more embodiments, the piston-needle assembly engages the plug to cause the plug to detach from the housing to allow the fluid preparation to flow from the reservoir through the opening into the chamber.

In one or more embodiments of any of the foregoing aspects, the valve member includes a piercing member and a seal, and wherein the seal is coupled to the housing at the opening. In one or more embodiments, the piston-needle assembly engages the piercing member to cause the piercing member to pierce the seal to allow the fluid preparation to flow from the reservoir through the opening into the chamber. In one or more embodiments, the piercing member is structured as a flexible tab with a tapered end. In one or more embodiments, the piercing member is structured as an elongated shaft with a tapered end.

In one or more embodiments of any of the foregoing aspects, the device further includes an ingestible enclosure for delivery into the GI tract of the subject.

In one or more embodiments of any of the foregoing aspects, the expandable member includes a balloon.

In one or more embodiments of any of the foregoing aspects, the device further includes a plurality of reactants disposed in the balloon for generating a gas pressure within the balloon to cause the balloon to expand within a GI lumen.

In one or more embodiments of any of the foregoing aspects, the piston-needle assembly is structured to move relative to the housing in response to the gas pressure applied against a surface of the piston-needle assembly to advance a portion of the piston-needle assembly into the GI lumen wall or surrounding tissue thereof.

In one or more embodiments of any of the foregoing aspects, the membrane includes a flexible material to allow for deformation of the membrane by the gas pressure applied against an outer surface of the membrane to expel the fluid preparation from the reservoir into the chamber.

In one or more embodiments of any of the foregoing aspects, the piston-needle assembly is structured to penetrate through the GI lumen wall into a peritoneum or peritoneal cavity of the subject for discharging the fluid preparation therein.

In one or more embodiments of any of the foregoing aspects, the reservoir defines a volume for containing up to about 400 μl of fluid.

In one or more embodiments of any of the foregoing aspects, the reservoir defines a volume for containing about 50 μl to about 300 μl of fluid.

In one or more embodiments of any of the foregoing aspects, the delivery assembly further includes a cover coupled to the housing, the cover defining a release mechanism for temporarily holding the piston-needle assembly at an axial position relative to the housing.

In one or more embodiments of any of the foregoing aspects, the cover includes a first cover section coupled to the housing and a second cover section coupled to the piston-needle assembly, and wherein the release mechanism is defined by one or more tabs detachably coupled between the first cover section and the second cover section.

In one or more embodiments of any of the foregoing aspects, the cover further includes one or more openings to allow a gas pressure to be applied against a surface of the piston-needle assembly, and wherein the one or more tabs are structured to detach from at least one of the first cover section or the second cover section to allow the piston-needle assembly to move axially relative to the housing in response to the gas pressure reaching a threshold value.

In one or more embodiments of any of the foregoing aspects, the membrane extends circumferentially about the housing.

In one or more embodiments of any of the foregoing aspects, the housing includes one or more fill ports for filling the reservoir with the fluid preparation.

In one or more embodiments of any of the foregoing aspects, the delivery assembly further includes a fluid preparation disposed in the reservoir, wherein the fluid preparation includes at least one therapeutic agent.

In one or more embodiments of any of the foregoing aspects, the at least one therapeutic agent is one or more selected from an immunosuppressive drug, a chemotherapy drug, a central nervous system (CNS) drug, an antidiabetic drug, an enzyme replacement therapy (ERT) drug, an antibody, a hormone, insulin, an incretin or a combination thereof, or an oligonucleotide.

The foregoing general description and following detailed description are provided by way of example and are intended to provide further explanation of the disclosure as claimed, without limiting the disclosure or the claims. Other objects, advantages, and novel features will be readily apparent to those skilled in the art from the following brief description of the drawings and detailed description of the disclosure.

### BRIEF DESCRIPTION OF THE DRAWINGS

FIG. 1 illustrates an embodiment of a device for delivering a fluid preparation into a GI tract of a subject in block diagrammatic form.

FIG. 2 illustrates a cross-sectional view of an embodiment of the device of FIG. 1.

FIG. 3 illustrates a detail view of a portion of the device of FIG. 2 including a delivery assembly.

FIG. 4 illustrates a partial perspective view of the delivery assembly of FIG. 3 without a membrane.

FIG. 5 illustrates another partial perspective view of the delivery assembly of FIG. 3 without a membrane.

FIG. 6 illustrates the device of FIG. 3 in a second state when the device has reached a desired location in the GI tract to deliver a fluid preparation.

FIG. 7 illustrates a perspective view of a valve member in the form of a piercing member according to an example embodiment.

FIG. 8 illustrates a partial cross-sectional view of the device of FIG. 2 with the piercing member of FIG. 7.

FIGS. 9-10 illustrate the device of FIG. 2 with a valve member in the form of a plug according to another example embodiment.

FIG. 11 illustrates a method of delivering a fluid preparation into the GI tract of a subject using the devices and assemblies of the present disclosure.

### DETAILED DESCRIPTION

Before discussing details of the devices, assemblies, and methods of the present disclosure, a few conventions are provided for the convenience of the reader.

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural references unless the context clearly dictates otherwise. Reference to an object in the singular is not intended to mean “one and only one” unless explicitly so stated, but rather “one or more.”

As used herein, a phrase in the form “A/B” or in the form “A and/or B” means (A), (B), or (A and B); a phrase in the form “at least one of A, B, or C” means (A), (B), (C), (A and B), (A and C), (B and C), or (A, B, and C).

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be omitted or can be used in another embodiment (e.g., in another embodiment illustrated and described herein, or in another embodiment within the scope of the present disclosure and not illustrated and/or not described herein).

The term “component” refers herein to one item of a set of one or more items that together make up a device, a composition, or a system under discussion. A component may be in a solid, powder, gel, plasma, fluid, gas, or other constitution. For example, a device may include multiple solid components which are assembled together to structure the device and may further include a fluid component that is disposed in the device. For another example, a composition may include a single component, or two or more components which are mixed together to make the composition. A composition may be in the form of a fluid, a slurry, a powder, or a solid (e.g., in a condensed or a consolidated form such as a tablet or microtablet). A device or system can include one or more compositions and/or one or more other components.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, pH, species, diet, health, gender, age, ancestry, disease, or tissue damage); it is to be understood that actual tolerances and environmental conditions before and/or after delivery can affect characteristics so that different components, devices, compositions, or systems with a same design can have different actual values with respect to those characteristics. Design encompasses also variations or modifications before or after manufacture.

The term “structured” or a grammatical variation thereof (e.g., “structure” or “structuring”) refers herein to a component, device, composition, or system that is manufactured according to a concept or design or variations thereof or modifications thereto (whether such variations or modifications occur before, during, or after manufacture) whether or not such concept or design is captured in a writing.

The term “body” refers herein to an animalia body, unless the context clearly dictates otherwise.

The term “subject” refers herein to a body into which an embodiment of the present disclosure is, or is intended to be, delivered. For example, with respect to humans, a subject may be a patient under treatment of a health care professional. The terms “individual,” “subject,” and “patient” may be used interchangeably herein, and refer to any individual animalia subject (e.g., bovine, canine, feline, equine, or human). In specific embodiments, the subject, individual, or patient is a human.

The term “fluid” refers herein to a liquid or gas, and encompasses moisture and humidity. The term “fluidic environment” refers herein to an environment in which one or more fluids are present.

The term “ingest” or a grammatical variation thereof (e.g., “ingesting”, “ingestion,” or “ingested”) refers herein to taking into the stomach, whether by swallowing or by other means of depositing into the stomach (e.g., by depositing into the stomach by endoscope or depositing into the stomach via a port).

The term “degrade” or a grammatical variation thereof (e.g., “degrading”, “degraded”, “degradable”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking. The term “non-degradable” refers to an expectation that degradation will be minimal, or within a certain acceptable design percentage, for at least an expected duration in an expected environment.

The term “degradation rate” or a grammatical variation thereof (e.g., “rate of degradation”) refers herein to a rate at which a material degrades. A designed degradation rate of a material in a particular implementation can be defined by a rate at which the material is expected to degrade under expected conditions (e.g., in physiological conditions) at a target delivery site. A designed degradation time for a particular implementation can refer to a designed time to complete degradation or a designed time to a partial degradation sufficient to accomplish a design purpose (e.g., breach). Accordingly, for example, a designed degradation time can be specific to a component and/or specific to expected conditions at a target delivery site. A designed degradation time can be short or long and can be defined in terms of approximate times, maximum times, or minimum times.

The term “substantially” is used herein to describe and account for small variations which may result from, for example, a manufacturing or assembly process. For example, when used in conjunction with a numerical value, the terms can refer to a variation in the value of less than or equal to ±10%.

The term “lumen” refers herein to the inside space of a tubular structure. Examples of lumens in a body include arteries, veins, and tubular cavities within organs.

The term “lumen wall” refers to a wall of a lumen, where the wall includes all layers from an inner perimeter to an outer perimeter of the lumen, such as, with respect to lumens in a body, the mucosa, submucosa, muscularis, serosa, and an outer wall of the lumen, with the constituent blood vessels and tissues.

The term “gastrointestinal tract” or “GI tract” refers herein to the intake/expulsion system of a body including, for example, the mouth, pharynx, esophagus, stomach, pylorus, small intestine, cecum, large intestine, colon, rectum, anus, and valves or sphincters therebetween.

The term “GI lumen” refers generally to any lumen of the GI tract (e.g., a lumen of the esophagus, stomach, small intestine, large intestine, or colon) and the term “GI lumen wall” refers to a lumen wall of a GI lumen.

As used herein, the terms “comprising”, “comprise”, “comprises”, “includes”, and “including” are intended to mean that the compositions and methods include the recited elements, but do not exclude others.

Referring generally to the Figures, disclosed herein are embodiments relating to devices, assemblies, and methods for delivering a fluid preparation into a lumen wall or surrounding tissue (e.g., a peritoneum or peritoneal cavity) of a GI tract of a subject. The devices are advantageously structured to substantially preserve one or more therapeutic agents in the fluid preparation within the GI tract for delivery into a GI lumen wall or surrounding tissue thereof. Delivery into the GI lumen wall or surrounding tissue can allow for systemic uptake of one or more therapeutic agents in the fluid preparation. Further, the fluid form of the preparation can allow for delivery of a broad range of dosages (e.g., up to about 100 mg or more) of one or more therapeutic agents for most therapeutic regimens. As discussed below, the disclosed devices and assemblies can deliver a variety of different types of therapeutic agents, such as macromolecules that are normally unsuitable for delivery by ingestion. In this manner, the disclosed devices, assemblies, and methods address many of the disadvantages associated with conventional oral and parenteral routes of administration.

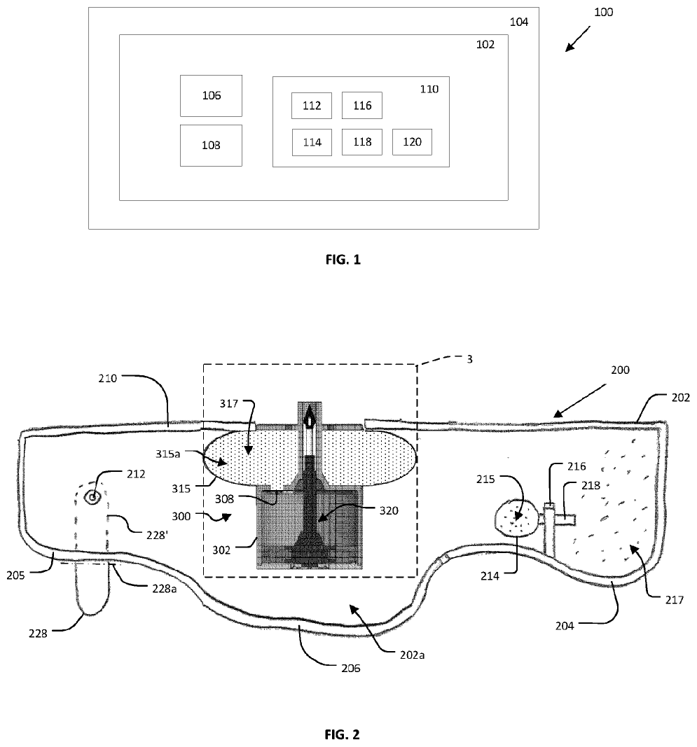

FIG. 1 illustrates in block diagrammatic form an example of a device 100 according to one or more embodiments of the present disclosure. Device 100 includes an enclosure 102, an optional outer coating 104, an expandable member 106, a release 108, and a delivery assembly 110. Delivery assembly 110 includes a housing 112, a membrane 114, a fluid preparation 116, a valve member 118, and a piston-needle assembly 120.

According to a non-limiting example, enclosure 102 (or optionally enclosure 102 and/or outer coating 104) can degrade at a desired location in the GI tract (e.g., the stomach or small intestine) of a subject for delivery of fluid preparation 116, as described in more detail below. As described in more detail below, in response to at least partial degradation of enclosure 102 and/or outer coating 104, release 108 is activated to cause a chemical reaction within expandable member 106 resulting in the formation of a gas to cause expandable member 106 to expand. Expansion of expandable member 106 within the GI lumen causes delivery assembly 110 to be positioned proximate to (e.g., adjacent or in contact with) the lumen wall. When the internal pressure generated by the gas within expandable member 106 reaches a threshold value (e.g., upon expansion of expandable member 106), the gas pressure causes piston-needle assembly 120 to move relative to housing 112 to advance a portion of piston-needle assembly 120 into the GI lumen wall. The internal pressure generated by the gas is also applied against an outer surface of membrane 114 before fluid preparation 116 is expelled into a chamber of housing 112 for subsequent delivery into the GI lumen wall. In response to sufficient movement of piston-needle assembly 120 (e.g., sufficient penetration into the GI lumen wall), valve member 118 opens to allow fluid preparation 116 to flow from a reservoir defined by membrane 114 and housing 112 into the chamber where fluid preparation 116 is routed through piston-needle assembly 120 and into the GI lumen wall or surrounding tissue thereof.

Enclosure 102 and outer coating 104, if present, are structured to allow for ingestion of device 100, and to temporarily protect the contents of device 100 from degradation within one or more portions of the GI tract of a subject. Enclosure 102 may take a variety of different forms and shapes, such as a swallowable capsule (e.g., a size 00 capsule, a size 000 capsule, or other size capsule), or any other structure that is suitable for ingestion by a subject and can house or contain one or more of the components of device 100 (e.g., at least member 106, release 108, and delivery assembly 110 as discussed below). In one or more embodiments, enclosure 102 includes two or more sections coupled (e.g., press-fit) together to define enclosure 102. For example, enclosure 102 may be structured as a capsule including a first section at least partially overlapping a second section in a press-fit arrangement to define enclosure 102. The first and second sections may be detachably coupled together so as to allow for separation of the two sections. In one or more embodiments, expandable member 106, release 108, and delivery assembly 110 are each structured to be contained within an interior of enclosure 102. Release 108 may optionally be located outside of the interior of enclosure 102, such as on an outer portion of enclosure 102.

In one or more embodiments, enclosure 102 can degrade under certain conditions. Further, different portions of enclosure 102 may be structured to degrade under different conditions or at different degradation rates depending on a target site within the GI tract for delivering fluid preparation 116. For example, a portion of, or all of, enclosure 102 may be constructed of a material that degrades in water (e.g., in the presence of water in the form of humidity or moisture in an ambient environment, such as within the body) and/or degrades when exposed to a pH level above a particular threshold or within a particular range (e.g., a pH level associated with a desired location or portion of the GI tract, such as a pH level associated with a target site within the GI tract for delivering fluid preparation 116).

Optional outer coating 104 optionally covers a portion of, or all of, enclosure 102. Outer coating 104 may include a single layer or multiple layers. The various layers may be formed of the same material or different materials. In one or more embodiments, outer coating 104 can degrade under certain conditions, as described above with reference to enclosure 102. An example of outer coating 104 is an enteric coating, such as an enteric coating that degrades in water at a given rate and/or degrades when exposed to solutions with a pH level above a particular threshold or within a particular range. Another example of outer coating 104 is a protective coating (e.g., wax), such as a coating which protects a portion of an outer surface of enclosure 102 from coming into contact with fluids or tissues (e.g., bodily tissue or fluids.)

In one or more embodiments, degradation of enclosure 102 and/or outer coating 104 allows fluid (e.g., bodily fluid in the stomach or in the intestine) to enter into an interior of enclosure 102/outer coating 104 to activate release 108. Alternatively, release 108 may be located on an outer portion of enclosure 102 and degradation of outer coating 104 may expose release 108 on a surface of enclosure 102 so as to facilitate activation of release 108. Alternatively, release 108 may be located on a portion of enclosure 102 without outer coating 104, and release 108 may be structured to degrade at a different rate and/or under different conditions in the GI tract than enclosure 102.

Enclosure 102 and/or outer coating 104 may define one or more degradation areas for localized degradation of enclosure 102 and/or outer coating 104 so as to, for example, allow for controlled degradation and separation of enclosure 102. For example, outer coating 104 may be selectively applied only to certain areas of enclosure 102 (e.g., on the ends of enclosure 102) so as to expose a selected portion of enclosure 102 (e.g., a middle portion of enclosure 102 between the ends), thereby defining an area of enclosure 102 that can degrade at a faster rate and/or degrade sooner than other areas of enclosure 102. This controlled degradation of enclosure 102 may allow for more consistent separation of enclosure 102 to thereby allow for delivery of fluid preparation 116 into the GI lumen wall.

Expandable member 106 is a flexible and adjustable structure. In one or more embodiments, expandable member 106 is structured to expand from a collapsed state (e.g., folded, rolled, flattened) to an expanded state within a desired location of a GI lumen for delivery of fluid preparation 116. Expandable member 106 may have a variety of different shapes, sizes, and configurations for being temporarily stored in enclosure 102 and for being deployed within a lumen of the GI tract of a subject. Expandable member 106 may be structured as a balloon, a bellows/accordion structure, or any other structure that can adjust from a collapsed state to an expanded state.

Expandable member 106 defines an interior volume for containing various components of device 100. For example, expandable member 106 may include (e.g., contain) one or more reactants that are temporarily separated from each other within the interior volume. The interior volume is structured to facilitate a chemical reaction caused by mixing the reactants together (e.g., in response to activation of release 108) to form a gas to cause expansion of expandable member 106. Expandable member 106 may further include a structure for deflating expandable member 106 upon delivery of fluid preparation 116 to facilitate excretion of expandable member 106 from a subject. Expandable member 106 is structured to stretch a defined amount without perforation upon expansion.

Expandable member 106 may be formed from one or more materials. Examples of suitable materials for expandable member 106 include hydroxypropyl methylcellulose (HPMC), polyvinyl acetate (PVA), lactide, glycolide, lactic acid, glycolic acid, par-dioxanone, trimethylene carbonate, caprolactone, and mixtures and copolymers thereof. Expandable member 106 may include one or more layers of material. Expandable member 106 may be a monolithic structure. In other embodiments, expandable member 106 may be composed of one or more sections that are coupled (e.g., sealed or sewn) together.

Release 108 is a chemical, mechanical, electrical, electro-mechanical, electro-chemical, chemo-mechanical, or electro-mechanical-chemical structure. Release 108 is structured to cause expansion of expandable member 106 upon activation of release 108. In one or more embodiments, release 108 is structured to be activated (e.g., degrade, release, move, open) in response to a condition in the GI tract. For example, release 108 may be structured to degrade in water, such that release 108 may degrade upon contact with fluid in the GI tract. For another example, release 108 may be structured to degrade at or above a particular pH level or within a range of pH levels associated with a location in the GI tract (e.g., a pH in the stomach, a pH in the intestine.) In these and other embodiments, release 108 may be made from a biodegradable material, such as an enteric material. Additionally or alternatively, release 108 may be structured as a latch, a clip, a cover, a plug, a coating, or any other structure that moves, opens, or otherwise releases in response to a condition in the GI tract. Release 108 may be formed from a single material or a combination of materials. Release 108 may include one or more components. In embodiments in which a plurality of components are included in release 108, the components may be co-located (e.g., co-axially) or may be physically separated from each other.

Delivery assembly 110 is a chemical, mechanical, electrical, electro-mechanical, electro-chemical, chemo-mechanical, or electro-mechanical-chemical structure. In one or more embodiments, delivery assembly 110 can include housing 112, membrane 114, fluid preparation 116, valve member 118, and piston-needle assembly 120. In other embodiments, delivery assembly 110 can include housing 112, membrane 114, fluid preparation 116, valve member 118, and a piston without a needle. In these embodiments, delivery assembly 110 is structured to deliver fluid preparation 116 as a fluidic jet from housing 112, where the fluidic jet has sufficient velocity to penetrate a GI lumen wall for systemic delivery of the one or more therapeutic agents contained in fluid preparation 116. Thus, where the discussion herein refers to piston-needle assembly 120, it should be understood that piston-needle assembly 120 could instead be a piston without a needle.

Delivery assembly 110 is structured to be coupled to, and disposed in (partially or fully), expandable member 106. In response to expansion of expandable member 106, delivery assembly 110 is further structured to deliver fluid preparation 116 into the GI lumen wall or surrounding tissue thereof via piston-needle assembly 120 or as a fluidic jet from housing 112. Device 100 may include one or more delivery assemblies 110 coupled to expandable member 106 to allow for delivery of one or more fluid preparations.

Housing 112 is structured to be coupled to, and is in fluid communication with, expandable member 106. Housing 112 defines one or more chambers for movably receiving piston-needle assembly 120. An outer portion of housing 112 defines a recess which, with membrane 114, cooperatively defines a reservoir for containing fluid preparation 116. Housing 112 includes one or more fill ports for filling reservoir with fluid preparation 116. Housing 112 is structured to allow gas pressure from within expandable member 106 to be applied against a surface of piston-needle assembly 120 to axially move piston-needle assembly 120 relative to housing 112. Housing 112 is further structured to interface with valve member 118 (e.g., via one or more openings/seals) to allow fluid preparation 116 to selectively flow from the reservoir into the one or more chambers where fluid preparation 116 is directed to piston-needle assembly 120 for delivery into the GI lumen wall or surrounding tissue. Housing 112 may include one or more vent ports to function as a pressure relief for a chamber of housing 112. Housing 112 may include one or more components. In embodiments in which a plurality of components are included in housing 112, the components may be co-located or may be physically separated from each other.

Membrane 114 is a flexible structure. Membrane 114 is coupled to housing 112. Membrane 114 and housing 112 cooperatively define a reservoir for containing fluid preparation 116. The reservoir may define a volume for containing up to about 400 μl or more of fluid, including 400 μl of fluid. The reservoir may define a volume for containing from about 50 μl to about 300 μl of fluid, including from 50 μl to 300 μl of fluid, such as 50 μl, 100 μl, 150 μl, 200 μl, 250 μl, or 300 μl of fluid, or any value therebetween, or may contain 300 μl, 350 μl, or 400 μl of fluid, or any value therebetween. Membrane 114 is further structured to expel fluid preparation 116 from the reservoir into one or more chambers of housing 112 in response to a pressure (e.g., from a gas generated within expandable member 106) applied against an outer surface of membrane 114.

Membrane 114 may have any suitable shape, such as torus, ellipsoidal, spherical, cuboidal, or other shape. Membrane 114 may be formed from, or otherwise include, a flexible or pliable material to allow for deformation (e.g., bending by pressure) of membrane 114 to expel fluid preparation 116. Examples of suitable materials for membrane 114 include a polymeric material (e.g., polyethylene terephthalate (PET)), or other flexible material having sufficiently low moisture and gas permeability properties for use with fluid preparation 116. Membrane 114 may be formed from a single material or a combination of materials. Further, membrane 114 may include one or more layers of a material. Membrane 114 may be a monolithic structure. In other embodiments, membrane 114 may be composed of multiple sections coupled (e.g., sealed or sewn) together to define membrane 114. Membrane 114 may include one or more components. In embodiments in which a plurality of components are included in membrane 114, the components may be co-located or may be physically separated from each other.

Fluid preparation 116 is a preparation including one or more components where the preparation is intended for a therapeutic, diagnostic, or other biological purpose. Fluid preparation 116 is in a fluid form, such as a liquid, a slurry, a gel, a suspension (including a colloidal suspension), a gas, a powder, or any combination thereof. Fluid preparation 116 includes one or more components including one or more therapeutic agents, such as a protein, a peptide, a polypeptide, an antibody, a drug (e.g., one or more selected from immunosuppressive drugs (e.g., adalimumab), chemotherapy drugs, central nervous system (CNS) drugs (e.g., antiparkinson agents, antiemetic agents), antidiabetic drugs (e.g., metformin), enzyme replacement therapy (ERT) drugs), hormones (e.g., parathyroid hormone (PTH), follicle stimulating hormone (FSH), and analogues thereof), insulin, an incretin or a combination thereof (e.g., GLP-1, GLP-2, GIP, glucagon, PYY, and analogues thereof)), an oligonucleotide (e.g., antisense oligonucleotides (ASO), RNA interference (RNAi), aptamer RNAs), a DNA or SiRNA transcript, a cell, a cytotoxic agent, a vaccine or other prophylactic agent, a nutraceutical agent, a vasodilator, or a vasoconstrictor, a delivery enhancing agent, a delay agent, an excipient, a diagnostic agent, or a substance for cosmetic enhancement. Fluid preparation 116 may include a therapeutically effective amount of one or more therapeutic agents as well as suitable amounts of other components (e.g., excipients) to achieve a desired therapeutic effect in a subject.

Valve member 118 is a mechanical structure for selectively permitting a flow of fluid preparation 116 from the reservoir into housing 112. Valve member 118 is structured to be disposed within a chamber of housing 112. Valve member 118 may be coupled to housing 112. In other embodiments, valve member 118 may be coupled to, or integrally formed with, piston-needle assembly 120. Valve member 118 is further structured to allow fluid preparation 116 to flow from the reservoir into a chamber of housing 112 in response to sufficient axial movement of piston-needle assembly 120. For example, housing 112 may include an opening extending between the reservoir and a piston chamber. A seal may be coupled at the opening to define a substantially fluid-tight seal between the reservoir and the piston chamber. Valve member 118 may be structured as a piercing member positioned within the piston chamber such that sufficient axial movement of piston-needle assembly 120 relative to housing 112 causes the piercing member to pierce the seal to allow fluid preparation 116 to flow from the reservoir into the piston chamber.

According to another example, valve member 118 may be structured as a plug detachably coupled to housing 112 at the opening. In this example, sufficient axial movement of piston-needle assembly 120 relative to housing 112 causes the plug to detach from housing 112 to provide a fluid pathway for fluid preparation 116 to flow from the reservoir into the piston chamber.

Valve member 118 may be formed from a single material or a combination of materials. Examples of suitable materials for valve member 118 include a polymeric material (e.g., Acrylonitrile Butadiene Styrene (ABS), Polyetheretherketone (PEEK)), silicone, a metal (e.g., stainless steel), or other material or combinations of materials. Valve member 118 may include one or more components. In embodiments in which a plurality of components are included in valve member 118, the components may be co-located or may be physically separated from each other.

Piston-needle assembly 120 is a mechanical or chemo-mechanical structure. Piston-needle assembly 120 is movably (e.g., slidably) disposed in housing 112. Piston-needle assembly 120 is structured to penetrate the GI lumen wall and to deliver fluid preparation 116 into the GI lumen wall or surrounding tissue thereof. Piston-needle assembly 120 may have a length sufficient to penetrate to a desired depth in a subject's GI lumen wall or surrounding tissue thereof. For example, piston-needle assembly 120 may have a length sufficient to penetrate through the GI lumen wall into a peritoneum or peritoneal cavity of a subject for discharging fluid preparation 116 therein.

Piston-needle assembly 120 may include one or more components. For example, piston-needle assembly 120 may include a piston, a shaft, and a needle. The piston-needle assembly 120 may include an inlet for receiving fluid preparation 116 from within the housing and a channel for directing fluid preparation 116 into the GI lumen wall.

The needle may be at least partially, or may be fully, degradable such that the needle can degrade within the GI lumen upon delivery of fluid preparation 116. Suitable materials for the needle may include, for example, polyethylene oxide (PEO), magnesium, or other materials or combinations of materials. Suitable materials for the piston and the shaft include, for example, a polymeric material (e.g., ABS), a metal (e.g., titanium), or other materials or combinations of materials. In other embodiments, the needle may be substantially non-degradable or may include substantially non-degradable portions. In any embodiments, and particularly in embodiments where the needle is substantially non-degradable, delivery assembly 110 may be equipped with a return spring or other structure to permit retraction of the needle from the GI lumen wall within housing 112 to prevent injury to a subject.

One or more components of device 100 (e.g., enclosure 102, outer coating 104, expandable member 106, release 108, delivery assembly 110) may be formed from, or otherwise include, one or more biodegradable materials to facilitate degradation of such components to, for example, allow for passage through the remainder of the intestinal tract of a subject after delivery of fluid preparation 116. Examples of biodegradable materials that may be suitable for use with various components of device 100 include, for example, hydroxypropyl methylcellulose (HPMC), polyvinyl acetate (PVA), lactide, glycolide, lactic acid, glycolic acid, par-dioxanone, trimethylene carbonate, caprolactone, and mixtures and copolymers thereof.

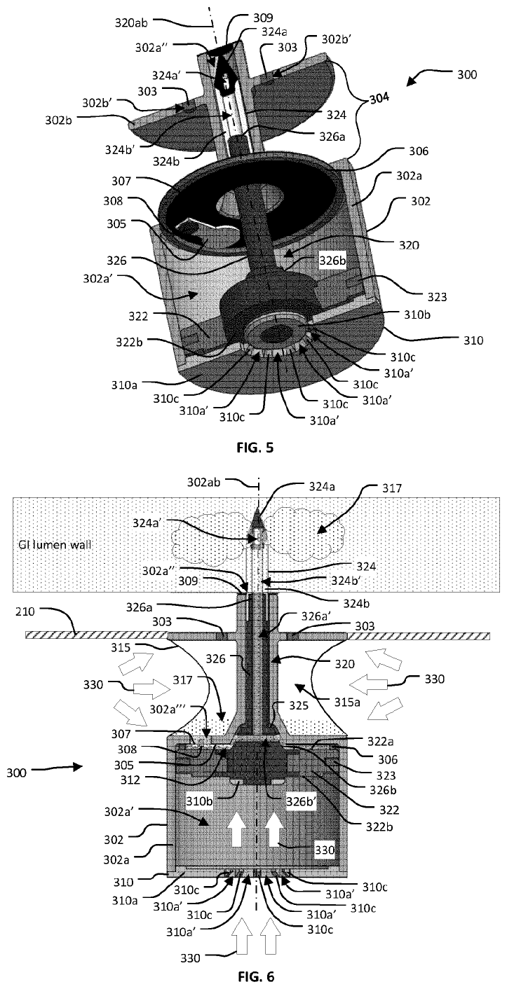

The preceding description is an overview of ingestible device 100. The following description with reference to FIGS. 2-12 relates to examples of various features and aspects of device 100. These examples are illustrative of, and not limiting on, device 100.

Referring to FIG. 2, a cross-sectional view of a device 200 (an embodiment of device 100) is illustrated in an unfolded and/or unrolled state. Device 200 is illustrated without enclosure 102 and optional outer coating 104. However, it should be appreciated that device 200 may be folded, rolled, and/or otherwise manipulated to be disposed within enclosure 102 (optionally provided with outer coating 104) to allow for ingestion of device 200 and subsequent delivery into the GI tract.

Device 200 includes an expandable member in the form of a balloon 202 (an embodiment of expandable member 106), a deflation valve 212, a reactant reservoir 214 containing a first reactant 215, a release 216 (an embodiment of release 108), a second reactant 217, and a delivery assembly 300 (an embodiment of delivery assembly 110.)

Still referring to FIG. 2, balloon 202 includes an inflator section 204, a deflator section 205, a lower section 206, and an elongated section 210 extending between inflator section 204 and deflator section 205. Lower section 206 extends downward (in the orientation shown in FIG. 2) between inflator section 204 and deflator section 205. The various sections of balloon 202 cooperatively define an interior 202a for containing various components of device 200. As discussed below, balloon 202 is structured to inflate at a desired location within a lumen of the GI tract (e.g., stomach, intestine) in response to the formation of a gas from a chemical reaction between first reactant 215 and second reactant 217 within interior 202a. In this way, balloon 202 can facilitate delivery of a fluid preparation 317 (an embodiment of fluid preparation 116) from delivery assembly 300 into the GI lumen wall or surrounding tissue thereof.

Balloon 202 has a size and shape to occupy a space in a GI lumen upon inflation of balloon 202 to allow for delivery of fluid preparation 317 into the GI lumen wall. For example, upon inflation of balloon 202, an outer periphery of balloon 202 (e.g., an outer periphery of lower section 206 and elongated section 210) pushes against a surface of the lumen wall. The pressure exerted by balloon 202 is sufficient to temporarily hold balloon 202 relative to the lumen wall for delivery of fluid preparation 317. Depending on an inner circumference of the lumen delivery site, lower section 206 may remain partially folded, or may extend fully, when balloon 202 is inflated. For example, if the lumen is relatively large and there is no obstruction to resist expansion of balloon 202, then balloon 202 would assume a fully inflated configuration with lower section 206 fully extended (as shown in FIG. 2.) If, however, the lumen is relatively small such that the inner lumen circumference is less than a maximum dimension of the fully inflated balloon 202, then lower section 206 would remain partially folded. In this way, balloon 202 can self-adjust to the size of a GI lumen to hold balloon 202 in position for delivery of fluid preparation 317, such that the same balloon 202 can be used for a broad range of lumen sizes (e.g., different inner circumferences.)

Deflation valve 212 is structured to cause deflation of balloon 202 upon completion of delivery of fluid preparation 317 into the GI lumen wall or surrounding tissue thereof. In this way, deflation valve 212 can facilitate passage of balloon 202 through the remainder of the GI tract to exit the anus of the subject. In the embodiment shown, deflation valve 212 is structured as a degradable plug which temporarily covers an opening leading into interior 202a. The degradable plug may be structured to degrade in response to contact with fluid in the GI tract (e.g., bodily fluid) to thereby allow gas contained in interior 202a to exit through the opening. For example, deflation valve 212 may be formed from, or include, an enteric material. Balloon 202 may include an optional flap 228 which can temporarily cover deflation valve 212 until balloon 202 is inflated to thereby prevent premature activation (e.g., degradation) of deflation valve 212. For example, flap 228 may be temporarily held (e.g., adhered, tacked, or otherwise held) in a folded position 228′ about a flap folding axis 228a. The expansion of balloon 202 can cause flap 228 to unfold from the folded position 228′ to expose deflation valve 212 and allow fluid in the GI tract to reach deflation valve 212 and cause its degradation, thereby providing an opening for gas to exit through thereby permitting deflation.

Deflation valve 212 is shown located on deflator section 205, but deflation valve 212 may be located elsewhere on expandable member 202 according to other embodiments. Further, balloon 202 may include more than one deflation valve 212. Deflation valve 212 may be structured differently than the embodiment shown in FIG. 2. For example, in other embodiments, deflation valve 212 may be structured as a degradable and/or movable cover disposed over an opening on balloon 202. In these embodiments, degradation and/or movement of the cover away from the opening can cause gas to exit from interior 202a through the opening, thereby permitting deflation.

Reactant reservoir 214 is disposed within interior 202a and is structured to hold first reactant 215 therein and to temporarily prevent first reactant 215 from contacting second reactant 217, which is separately disposed within interior 202a. First reactant 215 may be, for example, citric acid. Second reactant 217 may be, for example, a carbonate, such as potassium bicarbonate. In other embodiments, first reactant 215 and second reactant 217 may be other types of reactants (e.g., an acid and a base) which when mixed result in the formation of a gas sufficient to inflate balloon 202. Second reactant 217 is shown disposed within interior 202a at inflator section 204 near first reactant 215. In other embodiments, first reactant 215 and second reactant 217 may be contained in other areas of balloon 202 so long as they are temporarily separated from each other.

Reactant reservoir 214 defines an interior volume for containing first reactant 215. Reactant reservoir 214 may take a variety of different forms and shapes, such as a balloon or other structure. Reactant reservoir 214 is in selective fluid communication with interior 202a via reactant conduit 218 and release 216. Release 216 is coupled to reactant conduit 218 such that upon activation (e.g., degradation) of release 216, first reactant 215 can exit from reactant reservoir 214 into interior 202a via reactant conduit 218. For example, release 216 may be in the form of a degradable plug which blocks an interior portion of reactant conduit 218 to temporarily prevent first reactant 215 from entering interior 202a. When fluid in the GI tract contacts release 216 (e.g., upon degradation of enclosure 102 and/or outer coating 104), release 216 can subsequently degrade to allow reactant conduit 218 to discharge first reactant 215 into interior 202a.

In other embodiments, device 200 includes a clip, a band, or other structure for holding a portion of balloon 202 in such a manner so as to temporarily define separate chambers within interior 202a for separately containing first reactant 215 and second reactant 217, respectively. For example, a portion of balloon 202 may be pinched or compressed by a degradable clip or band to temporarily define the separate chambers within interior 202a. The chambers may be substantially sealed from each other to substantially prevent first reactant 215 and second reactant 217 from mixing. The clip or band may be located on an outer portion of balloon 202 such that exposure to fluid in the GI tract (e.g., upon degradation of enclosure 102 and/or outer coating 104) can cause degradation of the clip or band and subsequent release from balloon 202. Upon release of the clip or band or like structure from balloon 202, the separate chambers are no longer substantially sealed from each other thereby allowing first reactant 215 to mix with second reactant 217 within interior 202a.

Combining first reactant 215 with second reactant 217 within interior 202a causes a chemical reaction resulting in the formation of a gas (e.g., CO₂.) The gas causes balloon 202 to expand to an inflated state within a GI lumen resulting in substantial alignment of elongated section 210 with a surface of the GI lumen wall. Substantial alignment of elongated section 210 relative to the GI lumen wall can, advantageously, help to facilitate delivery of fluid preparation 317 from delivery assembly 300 into the GI lumen wall or surrounding tissue thereof.

Still referring to FIG. 2, delivery assembly 300 is coupled to balloon 202 and is partially disposed within interior 202a. The delivery assembly 300 is shown to include a housing 302 (an embodiment of housing 112), a piercing member 308 (an embodiment of valve member 118), a membrane 315 (an embodiment of membrane 114), fluid preparation 317 (an embodiment of fluid preparation 116), and a piston-needle assembly 320 (an embodiment of piston-needle assembly 120.) Membrane 315 and housing 302 cooperatively define a reservoir 315a for containing fluid preparation 317.

As discussed below, the gas that pressurizes balloon 202 can pass through one or more openings of housing 302 to apply a pressure against a surface of piston-needle assembly 320. When the pressure reaches a threshold value (e.g., upon full or partial inflation of balloon 202), the pressure causes piston-needle assembly 320 to move relative to housing 302 to advance a portion of piston-needle assembly 320 into the GI lumen wall or surrounding tissue thereof. The gas that pressurizes balloon 202 is also applied against an outer surface of membrane 315 for expelling fluid preparation 317 into housing 302. However, reservoir 315a is substantially fluidly sealed from housing 112 to prevent a flow of fluid preparation 317 from reservoir 315a to piston-needle assembly 320 until piston-needle assembly 320 has traveled a sufficient distance. Upon sufficient axial travel of piston-needle assembly 320 relative to housing 302 (e.g., upon penetrating the GI lumen wall), piercing member 308 is structured to pierce a seal on housing 302 to allow fluid preparation 317 to flow from reservoir 315a into housing 302 and through piston-needle assembly 320 for delivery into the GI lumen wall or surrounding tissue thereof.

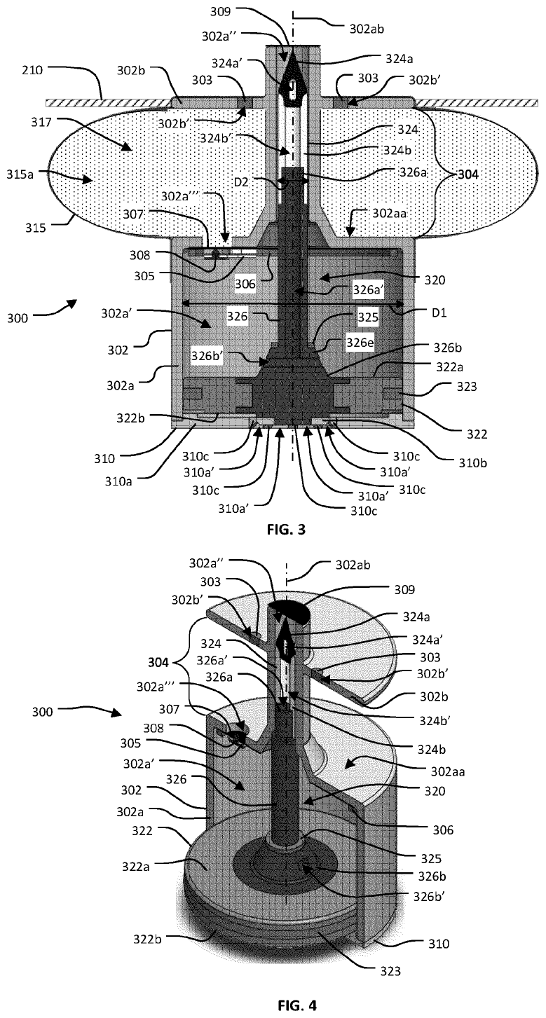

Referring to FIG. 3, a detail view of a portion of device 200 including delivery assembly 300 is illustrated. As shown in FIG. 3, delivery assembly 300 includes housing 302, an optional chamber seal 306, a reservoir seal 307, piercing member 308, a needle seal 309, a cover 310, membrane 315, fluid preparation 317, and piston-needle assembly 320. Piston-needle assembly 320 is collectively defined by a piston 322, a needle 324, and a shaft 326. In other embodiments, piston-needle assembly 320 is a monolithic structure including piston 322, needle 324, and shaft 326.

Referring to FIGS. 3-5, housing 302 includes a perimeter wall 302a which defines a piston chamber 302a′ and a needle chamber 302a″. Piston 322 is movably (e.g., slidably) disposed in piston chamber 302a′. Needle 324 and a portion of shaft 326 are disposed in needle chamber 302a″. In the orientation shown in FIG. 3, needle chamber 302a″ extends from an upper end of piston chamber 302a′. Piston chamber 302a′ has a substantially cylindrical shape defining a first inner diameter D1 which corresponds to an outer diameter of piston 322. Similarly, needle chamber 302a″ has a substantially cylindrical shape defining a second inner diameter D2 which corresponds to an outer diameter of needle 324 and shaft 326. It should be appreciated, however, that piston chamber 302a′ and needle chamber 302a″ may have different shapes besides cylindrical (e.g., ovular, spherical) according to other embodiments. As shown in FIG. 3, first inner diameter D1 is larger than second inner diameter D2 such that perimeter wall 302a defines a step region 302aa extending between an upper end of piston chamber 302a′ and a lower end of needle chamber 302a″. Piston chamber 302a′ and needle chamber 302a″ cooperatively define a longitudinal axis 302ab for piston-needle assembly 320 to move axially along.

Housing 302 further includes an upper wall 302b extending outwardly (e.g., radially) from an upper section of perimeter wall 302a which defines needle chamber 302a″. Housing 302 is structured to be coupled (e.g., heat sealed, adhered) to balloon 202 (e.g., to elongated section 210) at upper wall 302b. In an assembled state, a portion of perimeter wall 302a located above upper wall 302b extends outwardly from elongated section 210 of balloon 202 for positioning needle chamber 302a″ proximate to the GI lumen wall. Upper wall 302b and a portion of perimeter wall 302a (extending from upper wall 302b to step region 302aa) collectively define a recess 304 which extends circumferentially around needle chamber 302a″. As discussed below, membrane 315 is coupled to housing 302 such that membrane 315 encloses recess 304 to define reservoir 315a for containing fluid preparation 317 therein.

Upper wall 302b further defines one or more fill ports 302b′ for filling reservoir 315a with fluid preparation 317. For example, in the embodiment shown, upper wall 302b defines two fill ports 302b′ structured as through holes for filling (e.g., partially or fully) reservoir 315a with fluid preparation 317. After filling reservoir 315a, fill ports 302b′ are substantially fluidly sealed using separate seals 303 coupled thereto. Seals 303 may include, for example, silicone or aluminum foil. For example, seal 303 may include a tube inserted within each fill port 302b′ and a plug engaged with a side of the tube through a lateral opening of housing 302 to create a pinch-valve arrangement. In other embodiments, fill ports 302b′ may include a septum (e.g., silicone septum) such that fill ports 302b′ can self-seal after filling reservoir 315a with fluid preparation 317. In any case, reservoir 315a may be filled with fluid preparation 317 before delivery assembly 300 is coupled to balloon 202. This can, advantageously, allow for flexibility relating to aseptic assembly of device 200. For example, prior to coupling delivery assembly 310 to balloon 202, a vacuum may be applied to reservoir 315a via fill ports 302b′ in an aseptic environment to substantially evacuate reservoir 315a. The evacuated reservoir 315a can then be filled with fluid preparation 317 via fill ports 302b′ in the aseptic environment prior to assembly with balloon 202.

Still referring to FIGS. 3-5, perimeter wall 302a further defines an opening 302a′″ extending between reservoir 315a and piston chamber 302a′ at step region 302aa. Reservoir seal 307 is coupled (e.g., adhered) to perimeter wall 302a at opening 302a′″ to define a substantially fluid-tight seal between reservoir 315a and piston chamber 302a′. In this way, reservoir seal 307 substantially prevents fluid preparation 317 from entering piston chamber 302a′ through opening 302a′″. Reservoir seal 307 may be formed from a penetrable material such as aluminum foil.

Perimeter wall 302a and/or upper wall 302b may further define one or more vent ports for venting piston chamber 302a′ or balloon interior 202a during or after axial movement of piston-needle assembly 320. The vent ports may be in selective fluid communication with the GI lumen environment outside of device 200. Additionally or alternatively, perimeter wall 302a may include a ramp or other local projection extending from an inner surface thereof for pushing against piston seal 323 upon sufficient axial movement of piston 322 to create a fluid path through piston chamber 302a′ to allow for venting of balloon interior 202a to a GI lumen environment.

Piercing member 308 is disposed in piston chamber 302a′. In the embodiment shown in FIGS. 3-5, piercing member 308 is coupled to perimeter wall 302a adjacent to reservoir seal 307 by an adhesive tape 305 (e.g., polyimide tape). In other embodiments, piercing member 308 may be coupled to perimeter wall 302a by, for example, heat staking, ultrasonic welding, or other attachment means. In the embodiment shown in FIGS. 3-5, piercing member 308 is structured as an elongated shaft with a tapered end (e.g., pointed tip) similar to the structure of a tack. Piercing member 308 may be made from, for example, a polymeric material (e.g., polyether ether ketone (PEEK)), a metal (e.g., stainless steel), or other material or combinations of materials. In the embodiment shown in FIGS. 3-5, the tapered end of piercing member 308 is positioned adjacent the reservoir seal 307 by adhesive tape 305 attached to an inner surface of perimeter wall 302a. As discussed below with reference to FIG. 6, the tapered end is structured to pierce reservoir seal 307 in response to sufficient axial movement of piston-needle assembly 320 relative to housing 302 so as to cause fluid preparation 317 to flow from reservoir 315a into piston chamber 302a′.

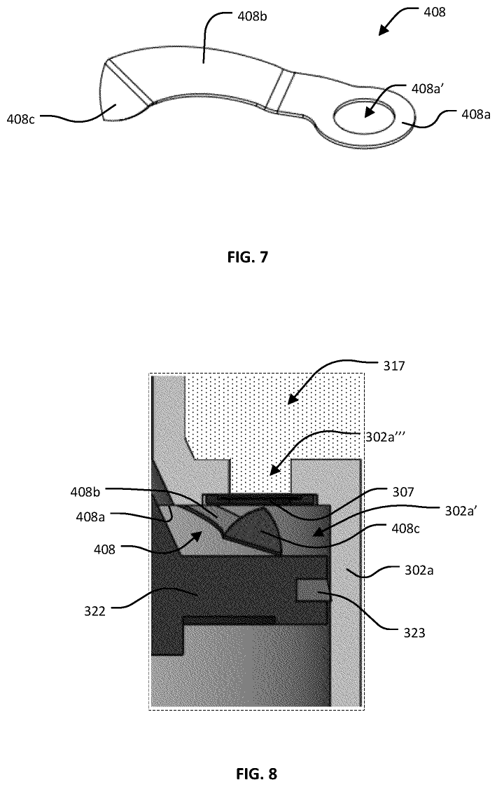

Another embodiment of valve member 118 is shown as a piercing member 408 in FIGS. 7-8. In this embodiment, piercing member 408 is structured as a flexible tab. Piercing member 408 may be made from, for example, a metal (e.g., stainless steel), a polymeric material (e.g., PEEK), or other material or combinations of materials. Piercing member 408 includes a planar end 408a defining an opening 408a′ for coupling piercing member 408 to perimeter wall 302a within piston chamber 302a′. For example, piercing member 408 may be heat staked, ultrasonically welded, adhered, or otherwise attached to perimeter wall 302a at planar end 408a. Piercing member 408 further includes an elongated section 408b extending away from planar end 408a. Elongated section 408b has a generally arcuate shape and extends downwardly (in the orientation shown in FIG. 8) at an acute angle relative to planar end 408a. The arcuate shape of elongated section 408b may help to avoid interference between piercing member 408 and opening 302a′″ upon piercing reservoir seal 307 to thereby allow for a substantially unobstructed flow of fluid preparation 317 into piston chamber 302a′. Elongated section 408b may also include one or more openings (e.g., slots) to further allow fluid preparation 317 to flow through opening 302a′″.

Piercing member 408 further includes a tapered section 408c extending from elongated section 408b. Tapered section 408c extends upwardly (in the orientation of FIG. 8) at an acute angle toward planar end 408a. Tapered section 408c terminates at a pointed end. Piercing member 408 is structured such that tapered section 408c can pierce reservoir seal 307 upon sufficient axial movement of piston-needle assembly 320 within piston chamber 302a′. For example, as shown in FIG. 8, when piston 322 moves axially within piston chamber 302a′, a portion of piston 322 can engage elongated section 408b to cause elongated section 408b to deform, or bend (elastically or permanently), relative to planar end 408. As a result, tapered section 408c can move toward reservoir seal 307 to pierce reservoir seal 307 and thereby allow fluid preparation 317 to flow through opening 302a′″ into piston chamber 302a′.

According to another embodiment, valve member 118 may be structured as a cartridge including a needle (or other feature having a pointed end) sufficient for piercing reservoir seal 307. The cartridge may be coupled to perimeter wall 302a at least partially within opening 302a′″ adjacent to reservoir seal 307. The cartridge may be structured such that when piston-needle assembly 320 engages the cartridge during axial movement of piston-needle assembly 320, the needle (or other feature) of the cartridge can move toward reservoir seal 307 to pierce reservoir seal 307.

According to another embodiment, valve member 118 may be defined by a portion of piston-needle assembly 320. For example, an upper portion of piston 322 may include a protrusion, or other structural feature, extending outwardly therefrom for piercing reservoir seal 307 upon piston-needle assembly 320 traveling a sufficient axial distance within piston chamber 302a′. The protrusion or other feature may be coupled to, or integrally formed with, piston 322 or shaft 326.

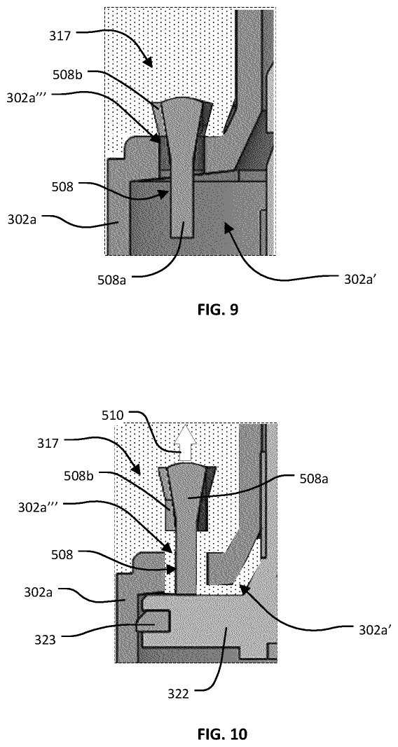

According to another embodiment shown in FIGS. 9-10, valve member 118 is in the form of a plug 508 instead of a piercing member. Plug 508 is detachably coupled to housing 302 at opening 302a″. Plug 508 is shown to include a body 508a and a seal 508b coupled to, or integrally formed with, body 508a. In other examples, plug 508 may be a unitary structure. In the example shown, seal 508b is structured to engage with housing 302 at opening 302a′″ to create a substantially fluid-tight seal therebetween. For example, seal 508b may be formed from or include a flexible polymeric material, such as silicone. Body 508a has an elongated structured with a tapered (e.g., frusto-conical) shape to help create an interference fit with housing 302 at opening 302a′″ to thereby retain plug 508 on housing 302. A portion of body 508a extends into piston chamber 302a′ to engage with a surface of piston 322 during axial movement of piston 322. In this way, piston 322 can force plug 508 to detach from housing 302 to provide a fluid pathway from reservoir 315a to piston chamber 302a′ through opening 302a′″ for discharging fluid preparation 317.

For example, as shown in FIG. 10, piston-needle assembly 320 has traveled a sufficient distance with piston chamber 302a′ such that an upper surface of piston 322 has engaged with body 508a of plug 508. As a result, plug 508 is forced out of opening 302a′″ away from housing 302, as indicated by unidirectional arrow 510 in FIG. 10, to thereby provide a fluid pathway from reservoir 315a to piston chamber 302a′ through opening 302a′″ for fluid preparation 317 to flow through.

Referring again to FIGS. 3-5, membrane 315 is coupled to housing 302 to define reservoir 315a. For example, membrane 315 may be coupled to housing 302 by heat sealing, ultrasonic welding, adhering, or by other means. In the embodiment shown, membrane 315 is coupled to an outer portion of upper wall 302b and an outer portion of perimeter wall 302a. Membrane 315 extends circumferentially about needle chamber 302a″ to enclose recess 304. In this way, membrane 315, perimeter wall 302a, and upper wall 302b cooperatively define reservoir 315a for containing fluid preparation 317 therein. Reservoir 315a may contain up to about 400 μl or more of fluid, including 400 μl of fluid. Reservoir 315a may contain from about 50 μl to about 300 μl of fluid, including from 50 μl to 300 μl of fluid, such as 50 μl, 100 μl, 150 μl, 200 μl, 250 μl, or 300 μl of fluid, or any value therebetween, or may contain 300 μl, 350 μl, or 400 μl of fluid, or any value therebetween. As discussed below, membrane 315 is structured to be deformed (e.g., compressed) by the gas pressure generated within balloon 202 (from reaction of first reactant 215 and second reactant 217) to expel fluid preparation 317 from reservoir 315a. Membrane 315 may have a relaxed shape or surface profile (i.e., when reservoir 315a is unfilled with fluid preparation 317) that is complementary to an outer surface profile of housing 302 which defines recess 304 such that membrane 315 can be sufficiently deformed to expel a substantial portion of fluid preparation 317 from reservoir 315a. Further, such a complementary shape of membrane 315 may help to facilitate sufficient evacuation of reservoir 315a for subsequent filling with fluid preparation 317 during assembly. Membrane 315 may be formed from a polymeric material (e.g., polyethylene terephthalate (PET)), or other flexible material or combinations of materials having sufficiently low moisture and gas permeability properties for use with fluid preparation 317.

Optionally, a membrane shield may be coupled to housing 302 to protect membrane 315 from, for example, potential damage during the assembly process of device 200. The membrane shield may substantially surround an outer surface of membrane 315. The membrane shield may be formed from a rigid or semi-rigid polymeric material and may define a plurality of openings to provide a fluid path for gas within interior 202a to be applied against an outer surface of membrane 315.

Piston-needle assembly 320 is disposed in housing 302 and is structured to move axially along longitudinal axis 302ab relative to housing 302. Piston-needle assembly 320 is collectively defined by at least piston 322, needle 324, and shaft 326. Piston-needle assembly 320 may further include a piston seal 323 and a shaft seal 325. In other embodiments, piston-needle assembly 320 is a monolithic structure including piston 322, needle 324, and shaft 326.

Piston 322 is movably (e.g., slidably) disposed in piston chamber 302a′. Piston 322 defines an upper surface 322a and a lower surface 322b. Piston 322 is temporarily held at an axial position within piston chamber 302a′ by a release mechanism defined by cover 310, the details of which are discussed below. Piston 322 is structured to move axially within piston chamber 302a′ in response to a threshold gas pressure applied against lower surface 322b within balloon 202. Piston seal 323 may be coupled to, or integrally formed with (e.g., insert molded), a peripheral side of piston 322 between upper surface 322a and lower surface 322b. Piston seal 323 is structured to engage an inner surface of perimeter wall 302a to thereby create a substantially fluid-tight seal between piston 322 and perimeter wall 302a within piston chamber 302a′. In this manner, piston seal 323 can help to substantially prevent fluid (e.g., air, fluid preparation 317) from entering or leaving the area of piston chamber 302a′ located above upper surface 322a. Piston seal 323 is further structured to allow for axial movement of piston 322 along longitudinal axis 302ab relative to perimeter wall 302a within piston chamber 302a′. Piston 322 may be formed from a polymeric material (e.g., acrylonitrile butadiene styrene (ABS)), or other material or combinations of materials. Piston seal 323 may be formed from silicone or other suitable material.

Needle 324 is coupled (e.g., press-fit) to, or integrally formed with (e.g., insert molded), shaft 326 and piston 322. In the unactuated state shown in FIG. 3, needle 324 is temporarily disposed within needle chamber 302a″. Needle seal 309 is coupled (e.g., adhered) to perimeter wall 302a at an upper end of needle chamber 302a″ to substantially prevent fluid in the GI tract from entering into needle chamber 302a″ until needle 324 pierces needle seal 309. Needle seal 309 may be formed from a penetrable material (e.g., aluminum foil) to allow needle 324 to pierce through needle seal 309. In this way, needle seal 309 and perimeter wall 302a can cooperatively define a substantially sterile environment within needle chamber 302a″ for containing needle 324 before delivery of fluid preparation 317 into the GI lumen wall or surrounding tissue.

As shown in FIGS. 3-5, needle 324 includes a tapered section 324a (e.g., pointed tip) that is structured to pierce though needle seal 309 and penetrate the GI lumen wall in response to sufficient axial movement of piston 322. Needle 324 further includes an elongated section 324b extending from tapered section 324a. Elongated section 324b may be coupled to, or integrally formed with, tapered section 324a. Elongated section 324b defines a needle channel 324b′ extending from needle opening 324a′ to an opposite end of elongated section 324b. As shown in FIG. 3, tapered section 324a defines a needle opening 324a′ extending through an end of tapered section 324a for discharging fluid preparation 317 into the GI lumen wall or surrounding tissue. Additionally or alternatively, elongated section 324b includes a side opening (e.g., slotted opening) for discharging fluid preparation 317 into the GI lumen wall or surrounding tissue. In some embodiments, needle 324 is at least partially, or may be fully, degradable such that at least a portion of needle 324 can substantially degrade within the GI lumen wall (or other location in the GI tract) upon delivery of fluid preparation 317. For example, needle 324 may be formed from, or otherwise include, polyethyleneoxide (PEO), magnesium, or other degradable material or combinations of materials.

In other embodiments, needle 324 may be formed from or otherwise include a substantially non-degradable material or combinations of materials, such as polyetheretherketone (PEEK), or other material or combinations of materials. In these and other embodiments, needle 324 may include a tip formed from a degradable material, such as magnesium, to allow for biodegradation of at least the tip within the body of the patient. In embodiments where needle 324 is substantially non-degradable or includes substantially non-degradable portions, device 200 is structured to retract needle 324 within housing 302 upon completing delivery of fluid preparation 317. For example, device 200 may include a return spring or other biasing member coupled to needle 324 for retracting needle 324 within housing 302 after delivery of fluid preparation 317.

Shaft 326 is coupled to (e.g., press-fit, adhered), or integrally formed with, piston 322 and needle 324. In one or more embodiments, shaft 326 and piston 322 may define a monolithic structure. In the embodiment shown, shaft 326 is structured as a substantially cylindrical member with a generally elongated configuration, although other shapes and configurations are contemplated according to other embodiments. Shaft 326 extends from a first end 326a to a second end 326b. First end 326a is coupled to (e.g., press-fit, adhered, insert molded), or integrally formed with, needle 324. Second end 326b is coupled to, or integrally formed with, piston 322. Shaft 326 further defines an inlet 326b′ for receiving fluid preparation 317 from piston chamber 302a′ and an inner shaft channel 326a′ for directing fluid preparation 317 from inlet 326b′ to needle channel 324b′. Inlet 326b′ extends radially from an outer surface of shaft 326 to inner shaft channel 326a′. Inner shaft channel 326a′ extends longitudinally along longitudinal axis 302ab. In other embodiments, shaft 326 may include a plurality of inlets and/or channels for directing fluid preparation 317 to needle channel 324b′.

Shaft seal 325 may be coupled to (e.g., press-fit, adhered), or integrally formed (e.g., insert molded) with, an outer portion of shaft 326. Shaft seal 325 is positioned on a circumferential ridge 326e of shaft 326 above inlet 326c. Shaft seal 325 is structured to engage with an inner surface of perimeter wall 302a between piston chamber 302a′ and needle chamber 302a″ to create a substantially fluid-tight seal between piston chamber 302a′ and needle chamber 302a″ upon sufficient axial travel of piston-needle assembly 320 relative to housing 302. In this manner, shaft seal 325 can help to prevent fluid preparation 317 from entering needle chamber 302a″ from piston chamber 302a′. Shaft seal 325 can also function to limit the axial travel of piston-needle assembly 320 within piston chamber 302a′ to help define a fluid channel 312 (see FIG. 6) between an upper surface of piston 322 and an inner surface of perimeter wall 302a within piston chamber 302a′. As discussed below with reference to FIG. 6, fluid channel 312 can function to direct fluid preparation 317 from opening 302a′″ to inlet 326c within piston chamber 302a′. In other embodiments, piston 322 may define a fluid channel in upper surface 322a for directing fluid preparation 317 toward inlet 326c.

In other embodiments, housing 302 and/or piston 322 may include one or more features (e.g., stand-off features, stop features, protrusions) to limit the axial travel of piston-needle assembly 320 within piston chamber 302a′ to help define fluid channel 312.

Delivery assembly 300 optionally includes lubricant seal 306 coupled to an inner surface of perimeter wall 302a within piston chamber 302a′. Lubricant seal 306 may help to minimize interaction between fluid preparation 317 and a lubricant that may be present within piston chamber 302a′ (e.g., on piston seal 323), which may help to minimize contamination of fluid preparation 317. As shown in FIG. 5, lubricant seal 306 extends circumferentially within piston chamber 302a′. In other embodiments, lubricant seal 306 is coupled to piston-needle assembly 320.

Cover 310 is coupled to housing 302 at an open end of piston chamber 302a′. Cover 310 is also detachably coupled to piston-needle assembly 320 by a release mechanism which temporarily holds piston-needle assembly 320 at an axial position within piston chamber 302a′. For example, in the embodiment shown in FIGS. 3-5, cover 310 includes a first cover section 310a coupled to perimeter wall 302a and a second cover section 302b coupled (e.g., heat staked, ultrasonically welded) to piston-needle assembly 320. First cover section 310a is detachably coupled to second cover section 310b by one or more tabs 310c which define the release mechanism. For example, tabs 310c may define a frangible connection between first cover section 310a and second cover section 310b.

As shown in FIG. 5, tabs 310c are spaced circumferentially around second cover section 310b and define a plurality of openings 310a′. Openings 310a′ can allow for a gas generated within interior 202a of balloon 202 to be applied against lower surface 322b of piston 322. According to other embodiments, cover 310 may include one or more openings disposed in other portions of cover 310. When the gas pressure applied against piston 322 reaches a threshold value, tabs 310c are structured to break away from at least one of the first cover section 310a or the second cover section 310b to thereby allow piston-needle assembly 320 to move axially along longitudinal axis 302ab within piston chamber 302a′ toward piercing member 308. The threshold pressure value may be a pressure associated with a fully inflated state of balloon 202 such that elongated section 210 is substantially aligned relative to the GI lumen wall before needle 324 is advanced into the GI lumen wall, e.g., before tabs 310c break away to allow piston-needle assembly 320 to move axially toward piercing member 308.

According to another embodiment, the release mechanism of cover 310 may be defined by one or more snap features which detachably couple cover 310 to piston-needle assembly 320. The one or more snap features may be structured to detach from piston-needle assembly 320 and/or from cover 310 in response to a threshold gas pressure applied against piston-needle assembly 320 to allow piston-needle assembly 320 to move axially away from cover 310 and toward piercing member 308 for advancement of needle 324 into the GI lumen wall, similar to tabs 310c described above.

According to another embodiment, the release mechanism of cover 310 may be defined by one or more flexible tabs extending from cover 310 toward longitudinal axis 302ab. The one or more flexible tabs may overlap with a complementary feature (e.g., a protrusion) on piston-needle assembly 320 to define an interference condition. In this embodiment, the one or more tabs may deflect in response to a threshold gas pressure applied against piston-needle assembly 320 to cause piston-needle assembly 320 to overcome the interference condition with the one or more tabs and allow piston-needle assembly 320 to move axially away from cover 310 and toward piercing member 308 for advancement of needle 324 into the GI lumen wall.

Referring to FIG. 6, a portion of device 200 including delivery assembly 300 is illustrated after device 200 has reached a desired location in the GI tract (e.g., the stomach or small intestine) for delivering fluid preparation 317. As shown in FIG. 6, balloon 202 has been inflated by a gas generated within interior 202a such that elongated section 210 is substantially aligned with the GI lumen wall. The generated gas within interior 202a applies a pressure (indicated by unidirectional arrows 330) against an outer surface of membrane 315 and against lower surface 322b of piston 322 through openings 310a′ of cover 310. When the gas pressure reaches a threshold value (e.g., a pressure associated with a fully or partly inflated state of balloon 202), the piston 322 detaches from cover 310 via detachable tabs 310c to allow piston-needle assembly 320 to move axially along longitudinal axis 302ab relative to housing 302 toward piercing member 308, such that needle 324 pierces needle seal 309 to penetrate the GI lumen wall. In this embodiment, fluid preparation 317 remains in reservoir 315a while the gas pressure is applied against an outer surface of membrane 315 until needle 324 sufficiently penetrates the GI lumen wall or surrounding tissue (e.g., to a desired penetration depth.)

Upon needle 324 sufficiently penetrating the GI lumen wall or surrounding tissue, piston 322 engages piercing member 308 to cause piercing member 308 to pierce reservoir seal 307. As a result, the gas pressure within interior 202a applied against an outer surface of membrane 315 causes fluid preparation 317 to flow from reservoir 315a into piston chamber 302a′ through opening 302a′″. Fluid preparation 317 is routed through fluid channel 312 to inlet 326b′ of shaft 326. Piston seal 323 and shaft seal 325 may help to contain fluid preparation 317 within fluid channel 312. The gas pressure applied against membrane 315 further causes fluid preparation 317 to flow through inlet 326b′, inner shaft channel 326a′, and needle channel 324b′ such that needle 324 discharges fluid preparation 317 from needle opening 324a′ into the GI lumen wall or surrounding tissue thereof. In this manner, one or more therapeutic agents contained in fluid preparation 317 can be delivered into the subject's blood stream.

In other embodiments, device 200 may be structured such that fluid preparation 317 is expelled into piston chamber 302a′ substantially simultaneously with needle 324 penetrating the GI lumen wall. For example, this may be achieved by selecting a length of needle 324, a length of shaft 326, a location of piercing member 308, a dimension of housing 302, or otherwise adjusting the amount of axial travel of piston-needle assembly 320 within piston chamber 302a′ such that the amount travel required to pierce seal 307 via piercing member 308 substantially corresponds with the amount of travel required for needle 324 to penetrate the GI lumen wall. Structuring device 200 such that piercing member 308 pierces seal 307 either after, or substantially simultaneously with, needle 324 penetrating the GI lumen wall can, advantageously, help to prevent discharge of fluid preparation 317 into the lumen environment to ensure delivery of a substantial portion of fluid preparation 317 into the GI lumen wall or surrounding tissue. In other words, device 200 can allow for sequential timing between penetrating the GI lumen wall and discharging fluid preparation 317 to substantially avoid discharging fluid preparation 317 into the lumen environment.

Upon completion of delivery of fluid preparation 317, needle 324 can subsequently degrade within the GI lumen wall, or other area within the GI tract, along with one or more additional components of device 200 (e.g., housing 302, membrane 315, shaft 326, piston 322, cover 310.) In embodiments where needle 324 is substantially non-degradable, device 200 is structured to retract needle 324 within housing 302 upon completing delivery of fluid preparation 317. For example, device 200 may include a return spring or other biasing member coupled to needle 324 for retracting needle 324 within housing 302 after delivery of fluid preparation 317. In either of these embodiments, deflation valve 212 can release a substantial amount of the gas contained within interior 202a to allow for substantial deflation of balloon 202 and subsequent traversal of device 200 through the remainder of the GI tract to exit the anus of the subject.

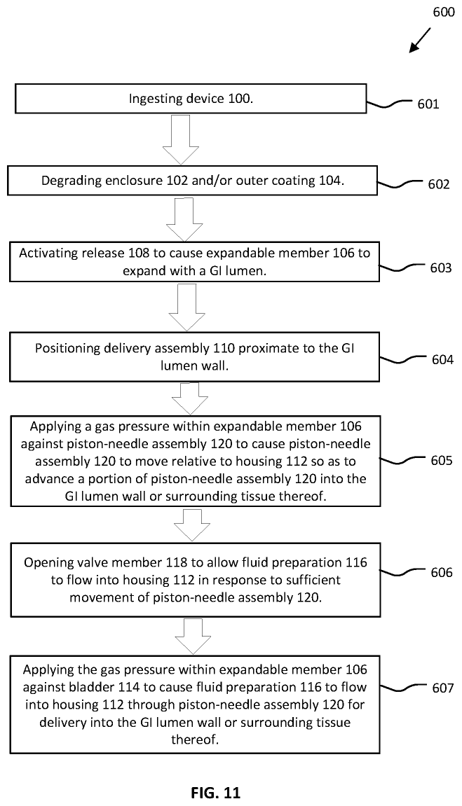

Referring now to FIG. 11, a method 600 of delivering a fluid preparation 116 into the GI tract of a subject using device 100 is illustrated. In a first step, the subject ingests device 100 (e.g., by swallowing device 100) (Step 601.) As a result of ingestion, enclosure 102 and/or outer coating 104 are at least partially (or may be fully) degraded upon device 100 reaching a desired location in the GI tract for delivery of fluid preparation 116 (Step 602.) In response to degradation of enclosure 102 and/or outer coating 104, release 108 is activated to cause expandable member 106 to expand within the GI lumen (Step 603.) Expansion of expandable member 106 within the GI lumen causes delivery assembly 110 to be positioned proximate to the GI lumen wall (Step 604.) When a gas pressure within expandable member 106 reaches a threshold value, the gas pressure causes piston-needle assembly 120 to move relative to housing 112 to advance a portion of piston-needle assembly 120 into the GI lumen wall or surrounding tissue thereof (Step 605.) Upon piston-needle assembly 120 traveling a sufficient axial distance relative to housing 112, valve member 118 is opened to allow fluid preparation 116 to flow into housing 112 (Step 606.) The gas pressure within expandable member 106 is applied against membrane 114 to expel fluid preparation 116 into housing 112 through piston-needle assembly 120 for delivery into the GI lumen wall or surrounding tissue thereof (Step 607.) In this manner, device 100 can deliver one or more therapeutic agents contained in fluid preparation 116 into the subject's blood stream for systemic delivery.

The foregoing description of various embodiments has been presented for purposes of illustration and description. It is not intended to limit the invention to the precise forms disclosed. Many modifications, variations and refinements will be apparent to practitioners skilled in the art. For example, embodiments of the device can be sized and otherwise adapted for various pediatric and neonatal applications as well as various veterinary applications. Also, those skilled in the art will recognize, or be able to ascertain using no more than routine experimentation, numerous equivalents to the specific devices and methods described herein. Such equivalents are considered to be within the scope of the present disclosure.

While the present disclosure has been described and illustrated with reference to specific embodiments thereof, these descriptions and illustrations do not limit the present disclosure. It can be clearly understood that various changes can be made, and equivalent components can be substituted within the embodiments, without departing from the true spirit and scope of the present disclosure as defined by the appended claims. Also, components, characteristics, or acts from one embodiment can be readily recombined or substituted with one or more components, characteristics or acts from other embodiments to form numerous additional embodiments within the scope of the invention. Moreover, components that are shown or described as being combined with other components, can, in various embodiments, exist as standalone components. Further, for any positive recitation of a component, characteristic, constituent, feature, step or the like, embodiments of the invention specifically contemplate the exclusion of that component, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it can be understood that these operations can be combined, sub-divided, or re-ordered to form an equivalent method without departing from the teachings of the present disclosure. Therefore, unless specifically indicated herein, the order and grouping of the operations are not limitations of the present disclosure.

1. A delivery assembly for an ingestible device for delivering a fluid preparation into a GI lumen wall or surrounding tissue thereof of a subject, the delivery assembly comprising:
a housing defining a chamber;
a piston-needle assembly movably disposed in the chamber, the piston-needle assembly comprising an inlet and a channel;
a membrane coupled to the housing such that the membrane and the housing cooperatively define a reservoir for containing the fluid preparation, wherein the housing further defines an opening extending between the chamber and the reservoir; and
a valve member for selectively controlling a flow of the fluid preparation from the reservoir to the chamber through the opening;
wherein in response to sufficient movement of the piston-needle assembly relative to the housing, the valve member is structured to open to allow the fluid preparation to flow from the reservoir through the opening into the chamber, the inlet, and the channel for delivery into the GI lumen wall or surrounding tissue thereof.

2. The delivery assembly of claim 1, wherein the piston-needle assembly and the housing cooperatively define a fluid channel within the chamber for directing the fluid preparation from the opening to the inlet.

3. The delivery assembly of claim 1, wherein the valve member comprises a plug detachably coupled to the housing at the opening.

4. The delivery assembly of claim 3, wherein the piston-needle assembly engages the plug to cause the plug to detach from the housing to allow the fluid preparation to flow from the reservoir through the opening into the chamber.

5. The delivery assembly of claim 1, wherein the valve member comprises a piercing member and a seal, and wherein the seal is coupled to the housing at the opening.

6. The delivery assembly of claim 5, wherein the piston-needle assembly engages the piercing member to cause the piercing member to pierce the seal to allow the fluid preparation to flow from the reservoir through the opening into the chamber.

7. The delivery assembly of claim 5, wherein the piercing member comprises a flexible tab with a tapered end.

8. The delivery assembly of claim 5, wherein the piercing member comprises an elongated shaft with a tapered end.

9. The delivery assembly of claim 1, wherein the delivery assembly is structured to be coupled to, and disposed in, an expandable member.

10. The delivery assembly of claim 1, wherein the delivery assembly is structured to be contained in an ingestible enclosure for delivery into the GI tract of the subject.

11. The delivery assembly of claim 1, wherein the piston-needle assembly is structured to move relative to the housing in response to a gas pressure applied against a surface of the piston-needle assembly to advance a portion of the piston-needle assembly into the GI lumen wall or surrounding tissue thereof.

12. The delivery assembly of claim 1, wherein the piston-needle assembly is structured to penetrate through the GI lumen wall into a peritoneum or peritoneal cavity of the subject for discharging the fluid preparation therein.

13. The delivery assembly of claim 1, wherein the reservoir defines a volume for containing up to about 400 μl of fluid.

14. The delivery assembly of claim 1, wherein the reservoir defines a volume for containing about 50 μl to about 300 μl of fluid.

15. The delivery assembly of claim 1, further comprising a cover coupled to the housing, the cover defining a release mechanism for temporarily holding the piston-needle assembly at an axial position relative to the housing.

16-17. (canceled)

18. The delivery assembly of claim 1, wherein the membrane extends circumferentially about the housing.

19. The delivery assembly of claim 1, wherein the membrane comprises a flexible material to allow for deformation of the membrane by a gas pressure applied against an outer surface of the membrane to expel the fluid preparation from the reservoir into the chamber.

20. The delivery assembly of claim 1, wherein the housing includes one or more fill ports for filling the reservoir with the fluid preparation.

21. The delivery assembly of claim 1, further comprising a fluid preparation disposed in the reservoir, wherein the fluid preparation comprises at least one therapeutic agent.

22. The delivery assembly of claim 21, wherein the at least one therapeutic agent is one or more selected from an immunosuppressive drug, a chemotherapy drug, a central nervous system (CNS) drug, an antidiabetic drug, an enzyme replacement therapy (ERT) drug, an antibody, a hormone, insulin, an incretin or a combination thereof, or an oligonucleotide.

23-47. (canceled)
